(19) United States
(12) Patent Application Publication
(10) Pub. No.: US 2008/0119714 A1
(43) Pub. Date: May 22, 2008

(54) Optimized clinical workflow method and apparatus for functional gastro-intestinal imaging

(76) Inventors: Oliver Meissner, Munich (DE); Michael Maschke, Lonnerstadt (DE)

Correspondence Address: BRINKS HOFER GILSON & LIONE, P.O. BOX 10395, CHICAGO, IL, 60610, US

(21) Appl. No.: 11/603,618

(22) Filed: Nov. 22, 2006

(51) Int. Cl. A61B 6/00; H05G 1/02

(52) U.S. Cl. 600/407; 378/196

(57) ABSTRACT

A treatment suite and method of use is described, having a digital imaging modality mounted to a first robot. A patient support apparatus mounted to a second robot. The robots cooperate to position a patient with respect to the imaging modality to obtain digital image data of an abdominal area so as to produce computed tomography images, including angiographic, soft tissue or hard tissue images. A third robot has a forcer configured to apply a force to a body part during the imaging process. Contrast agents may be administered during the imaging process. After diagnosis, the treatment of the patient may be performed without moving the patient from the patient support apparatus.

### TECHNICAL FIELD

The present application relates to an apparatus and method for assisting in the diagnosis or treatment of gastrointestinal illnesses.

### BACKGROUND

Gastrointestinal disorders represent an extremely inhomogeneous group of diseases. Among them are functional as well as inflammatory and cancerous diseases of the esophagus, stomach, small intestines, the colon or the rectum.

An imaging modality used in the evaluation of the abdomen is ultrasound (US), providing both morphological and functional information. Disadvantages of ultrasound, however, are operator dependency of performance and the difficulties in evaluating the intestines, especially in the case of an obstruction, diarrhea or flatulence. Computed tomography (CT) imaging and or magnetic resonance (MR) imaging can be considered state-of-the-art imaging for abdominal diseases, as they allow for cross-sectional or multidirectional imaging with high resolution and a large field of view. With the use of intravascular as well as intra-intestinal contrast material, CT/MR not only obtains information about the lumen, but also on surrounding tissues of the gastrointestinal tract. CT or MR colonography allows for an interior view of the intestines that can otherwise only be seen using an endoscope inserted into the rectum.

CT/MR colonography is used to screen for polyps in the colon and rectum. Polyps are benign growths that arise from the inner lining of the intestine; some polyps may grow and turn into cancers. A goal of screening is to find these growths in the early stages, so they can be removed before cancer has a chance to develop. With CT/MR, direct intervention such as the abrasion of polyps is not possible. Additionally, functional information such as the evaluation of compressibility of inflamed bowel loops, accelerated or delayed digestion or limited movement into an adhesion of bowel loops is not available with CT or MR. Furthermore, complex examinations, such as defecography, cannot optimally be performed in a supine position.

A barium enema is an examination of the large bowel, looking for abnormalities such as diverticula, polyps and cancer. Because of an ability to detect fine mucosal detail, double-contrast barium studies are used for diagnosing early stages of inflammatory and cancerous diseases like ulcerative colitis, Crohn's disease or colon/rectal cancer. An esophagogram is an imaging test to evaluate the swallowing function from the mouth to the stomach. During this examination, it is necessary to bring the patient to an upright or head tilt position.

The diagnosis of patients suspected of having gastrointestinal disorders is complex and often time-consuming. Normally, a typical clinical workflow consists of the following steps: clinical examination, transfer to the ultrasound department to perform abdominal sonography (US); and, transfer to CT or MR department to perform CT or MR of the abdomen. In the case that these tests are inconclusive, the patient may be transferred to the radiological department to perform radiographic examinations such as an esophagogram, barium enema, or defecogram. Where intervention is necessary, the patient may be treated with minimally invasive techniques or by surgery in another treatment room.

Flouroscopic systems, such as the Sireskop SD from Siemens AG do not provide 3-D imaging or do not have sufficient patient access for performing complex procedures. Angiographic systems, e.g. Axiom DynaCT from Siemens AG can form 3-D images with 3-D soft-tissue image synthesis, but have only limited capability to perform examination on patients in an upright or tilted position.

### SUMMARY

A treatment unit is described, including a robot. An imaging modality, capable of obtaining digital imaging data of a patient, is manipulably mounted to the a first robot. A patient support apparatus, capable of supporting a patient is manipulably mounted to the second robot. A controller is configured to cause the first robot and the second robot to cooperatively orient a patient such that digital image data of a desired portion of a patient may be obtained by the digital imaging modality, and computed tomographic images are synthesized from the digital image data. A third robot may be configured and controlled to apply a force to a body part of the patient when the digital image data is being obtained, and the sequence and amount of force coordinate with the imaging program. Any of the first, second and third robots may be the same robot.

Alternatively, the patient support apparatus may be a patient examination table, or the like, mountable to a pole, plinth, or holding device. A robot may place an imaging modality such a C-arm X-ray device on a first holding device capable of temporarily securing the imaging modality. The robot may temporarily attach to the patient support apparatus and position the support apparatus securing the patient in a suitable position for examination treatment of the patient and then secure the patient support apparatus to a second holding device. Subsequently, the robot may retrieve the imaging modality from the first holding device and the system may be operated to perform the imaging functions.

A method of diagnosing or treating a patient is described, including the steps of providing a digital imaging modality and mounting the digital imaging modality to a first robot, which may be mounted to a surface of a room. The method further includes providing a patient support apparatus and mounting the patient support apparatus to a second robot. After the patient is placed on the patient support apparatus, the robots are controlled for cooperatively controlling the digital imaging modality and the patient secured to the patient support apparatus such that a desired set of imaging data may be obtained for a portion of a patient, such as the abdomen, and the computing a computed tomography (CT) image using the data. When a diagnosis has been made and a treatment protocol has been determined, a direct intervention treatment protocol may be initiated while maintaining the patient on the patient support apparatus. In another aspect, the method may include controlling a third robot such that a force is applied to a part of the patient during the time that the imaging process is being performed. The third robot may be mounted to the first or second robot, or to the patient support apparatus, or separately mounted.

### BRIEF DESCRIPTION OF THE DRAWINGS

FIG. 1 is a block diagram of a treatment equipment suite;

FIG. 2 is a perspective view of a treatment room; and

FIG. 3 is a flow diagram of an example of a diagnosis and treatment work flow.

### DETAILED DESCRIPTION

Exemplary embodiments may be better understood with reference to the drawings. Like numbered elements in the same or different drawings perform equivalent functions.

In the interest of clarity, not all the routine features of the examples herein are described. It will of course be appreciated that in the development of any such actual implementation, numerous implementation-specific decisions must be made to achieve a developers' specific goals, such as compliance with system and business related constraints, and that these goals will vary from one implementation to another.

A gastroenterology diagnosis or treatment room (hereinafter “treatment room”) in which the patient need not be transported from apparatus-to-apparatus or room-to-room between the individual steps in diagnosis and therapy, and a method of use of the treatment room, is described. The imaging modality used in the treatment room may be a C-arm X-ray unit or other imaging modalities, such as an ultrasound device, or the like, or later developed imaging technologies capable of acquiring data for three-dimensional (3D) imaging.

The combination of hardware and software to accomplish the tasks described herein may be termed a platform or “therapy unit”. The instructions for implementing processes of the platform may be provided on computer-readable storage media or memories, such as a cache, buffer, RAM, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

The instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a given computer, system, or device.

Where the term “data network”, “web” or “Internet” is used, the intent is to describe an internetworking environment, which may include both local and wide area networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths.

Communications between the devices, systems and applications may be by the use of either wired or wireless connections. Wireless communication may include, audio, radio, lightwave or other technique not requiring a physical connection between a transmitting device and a corresponding receiving device. While the communication is described as being from a transmitter to a receiver, this does not exclude the reverse path, and a wireless communications device may include both transmitting and receiving functions.

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

A method of diagnosis and treatment of gastrointestinal disorders includes: providing an imaging modality capable of collecting data suitable for synthesis of three-dimensional (3D) images of the interior of a patient, the imaging modality mountable to a first robot; providing a patient support apparatus, which may be a type of gurney or examination table, which may have the capability of captivating the patient and may be mounted to a second robotic device. The first robotic device and the second robotic device may be the same robot, and the imaging modality and the patient support apparatus may be detachably mounted to the robot. The robotic device may also be provided with the capability for transferring the patient from a bed or gurney to the patient support apparatus, or may cooperate with another robot to perform such a function. The robots may be controlled in accordance with a predetermined diagnostic program or by manual input from an operator so that the imaging modality mounted to a first robotic device may be positioned with respect to the patient. The patient positioning may be varied so that the plane of the patient is horizontal, vertical, or disposed at an angle therebetween, depending on the examination or treatment protocol being performed.

The method may further comprise operating the first robot and an imaging modality attached thereto to obtain imaging data suitable for synthesis of 3D images. An arc of between about 180 degrees and about 360 degrees may be traversed by robotically rotating a C-arm X-ray device about an axis perpendicular to the principal axis of radiation. An electrocardiogram (EKG) or a respiratory monitor may be used for coordinating the motion of the C-arm and the timing of image acquisition with patient bodily functions such as heartbeat or breathing. Alternatively, such sensors may be used for selecting the data from the data base of acquired data for improving improve image quality by using data from a same phase of a bodily function.

The method may further include the acquisition of data by the imaging modality to obtain computed-tomography-like (CT-like) data suitable for reconstruction of the whole or part of the abdomen in conjunction with other diagnostic devices or aids.

For example, cross-sectional images of the abdomen may be synthesized after administration of intravenous contrast; virtual reconstruction of the esophagus or the stomach after administration of an intraluminal contrast material such as GASTROGRAFIN (Schering AG, Berlin, Germany),virtual colonography with 3D reconstruction after administration of intra-luminal contrast material, such as a barium enema; or, imaging of the intra-abdominal vessels after intravenous administration of contrast material. A time-series of image data sets may be obtained so as to study the time history of the diffusion of the contrast material, and more than one contrast material, such as vascular or barium contrast material may be administered during a session. It may be possible to administer contrast agents contemporaneously, taking account of the differing diffusion rates thereof, and perform several examination protocols in a continuous session.

Inter-arterial angiography can also be performed in situations where arterial/venous disease is possible (e.g., mesenteric ischemia, infarction, thrombosis)

Examples of imaging studies that may be performed using the treatment suite and method described herein include, but are not limited to: esophagography, complete work up of the stomach, examination of the small intestine, barium enema of the large intestines and defecography. Based on the results of the diagnosis, a direct interventional procedure may be performed, such as abrasion of suspicious polyps using an endoscope, vascular intervention, or the like. The method may therefore include orientating the patient in an optimal position for each stage of the diagnosis and treatment, such as in upright, head-tilt, prone or sitting positions.

In another aspect, the method may include manipulating of the patient using robotic or ancillary devices. For example, it may be desirable to provide for manual or programmed compression of bowel loops. A robotic attachment may provided to the patient support apparatus or the imaging apparatus or the robots, so that compressions may be performed using an ancillary robotic arm. In such a circumstance the control unit serves to maintain the current position status of the robots and attachments thereto, preventing collisions between the mechanical parts, and of the apparatus with the patient. The method may further include coordinating the action of the compression device with the recording of image data.

In an example, a patient is placed or secured to a patient support apparatus and administrated one or more contrast media such as barium or GASTROGRAFIN, either orally or rectally, and the patient may be positioned as appropriate for the procedure to be performed. A colonographic morphological image may be obtained which may be a cross-section, 3D reconstruction, or virtual colonoscopy. The examination also may include diagnostic intra-arterial angiography. The patient may be further repositioned to any required position, such as head-up or down, supine, prone, standing, or sitting, and fluoroscopic images of the gastrointestinal tract obtained so as to produce an esophagogram, a defecogram, or other image type.

In another aspect, the images may be used for diagnosing the disorder and, when appropriate, initiating the indicated treatment protocol, which may be performed With the aid of the apparatus of the treatment suite.

The treatment room may have an imaging modality such as an X-ray source and an X-ray detector. The X-ray source may be mounted to a first robot and the X-ray detector to a second robot. In an alternative, the X-ray source may be fastened to a C-arm, U-bracket, or the like, jointly with the X-ray detector, and may be secured to a robot. Robots may be fixedly mounted to a ceiling, wall or floor, or be guided in the one or more dimensions on a rail structure to which the robot may be captivated, or the like.

Further, the treatment suite may have a patient support apparatus such as stretcher or gurney, or provisions for mounting the same. The patient support apparatus may be made of materials that may be substantially transparent to X-rays and may be positionable manually or by a motor or hydraulic mechanism in various coordinate orientations such as height, longitudinal, transverse or rotational directions; and, may be inclined in any of the coordinate directions, rotated about a center point, or execute circular, elliptical or other rotary motions about a specified point or in a specified plane. The patient support apparatus may be mounted to a second robot, which may be mounted to a floor, a wall or a ceiling. When the robot is mounted to the floor, the robot may move freely in the horizontal direction, being held in contact with the floor by the force of gravity. The robot may be movable with respect to a surface such as the floor so as to facilitate transferring the patient between treatment stations or rooms. The robot may further be capable of transferring the patient to another patient support apparatus, such as an operating table, a bed, or the like. Alternatively, the robot may be guided by rails or the like.

The imaging modality of the diagnostic device may further comprise an X-ray tube, high-voltage power supply, radiation aperture, X-ray detector, digital imaging system, system controller, as well as user control and display units. The X-ray detectors, may be amorphous Selenium (a-Se), PbI2, CdTe or HgI2 detectors using direct detection and TFT technology, or indirect detectors as is known in the art, or may be subsequently be developed, to provide high resolution, high-dynamic-range real-time X-ray detection. The X-ray source may be rotated around the patient along a circular or elliptical path. The X-ray detector may be disposed diametrically opposed to the X-ray source and such that the plane of the detector is perpendicular to the axis of the X-ray source. This orientation may, for example, be maintained by attaching the X-ray source and X-ray detector to a C-arm, a U-arm or the like.

The imaging device is operated by rotating, for example, the C-arm such that the opposed X-ray source and X-ray detector traverse an angular range of at least about 180 degrees about an axis perpendicular to the plane of the C-arm. A 3D image may be reconstructed from the detected X-ray data. For example, a soft tissue image may be reconstructed using the methods described in US Pg-Pub US 2006/0120507 entitled “Angiographic X-ray Diagnostic Device for Rotational Angiography, filed on Nov. 21, 2005”, which is incorporated herein by reference. The algorithmic and measurement aspects of computed tomography images are being improved, and the processing of the images obtained by the imaging devices are expected to continue to improve in resolution and dynamic range, speed, and in reduction of the X-ray dosage.

The term “X-ray” is used to describe any device that uses ionizing radiation to obtain data regarding the opacity of a path through a patient, regardless of the wavelength of the radiation used.

Image quality may be improved by the use of an electrocardiogram (EKG) or respiration-controlled processing of the 2-D projection images used for the synthesis of 3D CT images, or for 4D images (that is, time varying 3D images). One method of using bodily function monitors such as an EKG or respiration monitor is to select the images to be used in the synthesis of a 3D image from portions of the image data set corresponding to similar stages of a heart or respiration cycle. Alternatively, the bodily function monitor may control the movement of the C-arm and the time of obtaining the image data. Such digital processes do not, however, exclude the use of X-ray film (in an X-ray cassette).

Where the operation of the treatment suite and the method makes used of automated or machine guided movements of the various pieces of examination or treatment apparatus, a collision avoidance system may be used to prevent injury to the patient or apparatus by determining the relative positions of the equipment pieces with respect to each other and to the patient. Such a collision avoidance system may use ancillary equipment such as ultrasonic or optical sensors, determinations of the equipment locations from the equipment controls and the system knowledge of the equipment positions relative to a baseline. The combination of hardware and software is used is intended to maintain a knowledge data base of the spatial location of the various objects to avoid unwanted contact therebetween, and to immobilize the equipment when a dangerous situation is encountered. This action may be preceded by one or more types of warning messages or sounds.

User control units may include provision for a selection of examination or treatment options. If an examination program for the stomach is selected, for example, the system components, image processing and the associated emitters, detectors and patient support apparatus positions may be automatically adjusted by a system controller to be positioned and configured to perform the predetermined examination protocol.

The examination and treatment suite may include a DICOM (Digital Communication in Medicine) interface including MPPS (Modality Performed Procedure Step), having the capability of further processing the image information and patient data, and interfacing with a data network.

A treatment suite may have additional treatment and diagnostic equipment such as a ventilator, a patient monitor, a mobile medication and instrument cabinet, a data terminal for inputting and outputting patient data, such as demographic data, insurance card, laboratory data, patient history and diagnosis information (for example, in the form of a “wireless notebook PC” or the like), various video displays for displaying data and images, and a digital camera unit for monitoring and video documentation of the individual diagnostic and therapeutic steps.

When a patient is brought to the treatment room, the robotic aspects of the system may be used to efficiently transfer and orient the patient in accordance with the selected diagnostic or treatment protocol, or in response to an operator control input. A robotic arm may facilitate rapid and precise positioning of an imaging device such as the C-arm X-ray device.

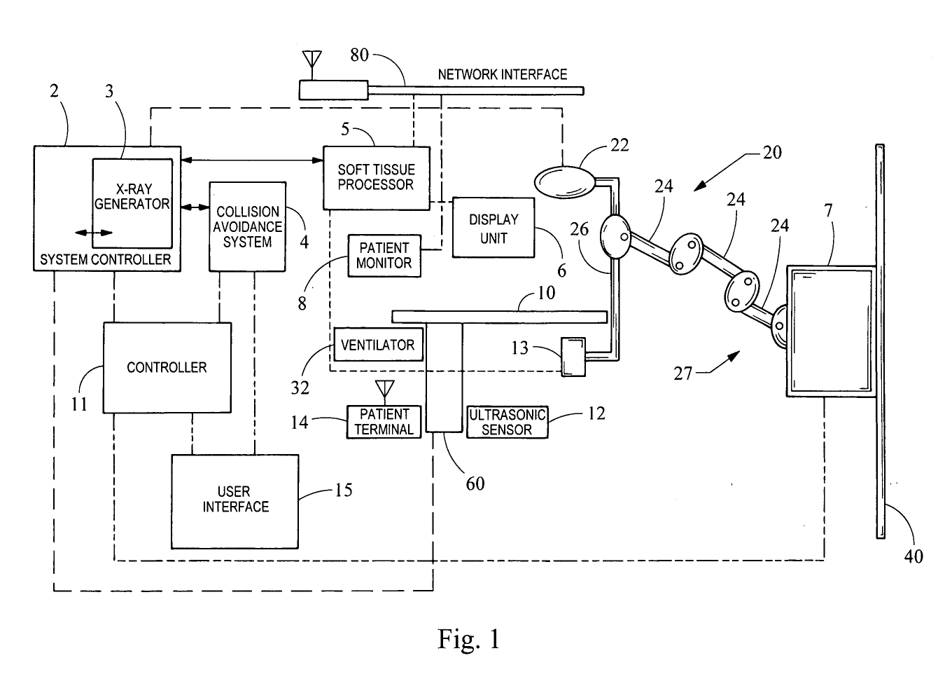

FIG. 1 shows a block diagram of an example of a treatment suite. Other embodiments of the treatment suite may include fewer than all of the devices, or functions, shown in FIG. 1. A C-arm X-ray device 20 is representative of an imaging modality which may be used and comprises a C-arm support 26 to which an X-ray source 22, which may include a diaphragm to limit the field of view, and an X-ray detector 13 may be mounted so as to face each other along a central axis of radiation. The C-arm 26 is mounted to a robotic device 27 comprising a mounting device 7, and one or more arms 24 which are articulated so as to be capable of positioning the C-arm X-ray device with respect to a patient support apparatus 10. The robotic device 27 may be controlled by a control unit 11, which may send commands causing a motive device (not shown) to move the arms 24. The motive device may be a motor or a hydraulic mechanism. The mounting device may be mounted to a wall 40 as shown, to a ceiling or to a floor, and may be capable of moving in longitudinal and transverse directions with respect to the mounting surface.

The C-arm X-ray device 20 is rotatable such that a sequence of projection X-ray images is obtained by an X-ray detector 13 positioned on an opposite side of the patient from the X-ray source 22, and the images are reconstructed by any technique of processing for realizing computed tomographic (CT) images. The patient is not shown in this figure, but would be positioned on the patient support apparatus 10. The patient support apparatus 10 may be a stretcher, gurney or the like attached to a robot 60 having similar characteristics to the robot 20 associated with the X-ray device 20. The patient support apparatus 10 may also be attached to a fixed support or adapted to be removably attached to the robot.

The patient may be secured to the patient support apparatus 10 so that the robot 60 may position and reposition the patient during the course of examination, diagnosis or treatment, so as to minimize the time required for the connection and disconnection of ancillary equipment and life support devices, or to permit the equipment and devices to remain connected for all of or a portion of the diagnosis and treatment process. Aspects of the patient support apparatus 10 may be manipulable by the robot 60, or manually, in order to position the patient for procedures where the patient may be in a sitting or upright position.

Additional, different, or fewer components may be provided. The devices and functions shown are representative, but not inclusive. The individual units, devices, or functions may communicate with each other over cables or in a wireless manner, and the use of dashed lines of different types for some of the connections in FIG. 1 is intended to suggest that alternative means of connectivity may be used.

The C-arm X-ray radiographic unit and the associated image processing may produce angiographic and soft tissue computed tomographic images comparable to, for example, CT equipment, while permitting more convenient access to the patient for ancillary equipment and treatment procedures. A separate processor 5 may be provided for this purpose, or the function may be combined with other processing functions.

An ultrasonic sensor 12 may be provided on one or more of the devices in the equipment suite, so as to measure the relative distance between, for example, parts of the C-arm X-ray device 20 and the patient support apparatus 10, or other equipment so as to aid in avoiding undesired contact between the devices, or contact between the devices and the patient. The relative position data and the ultrasonic data may be communicated to a collision processing system 4 which may be configured to prevent unsafe positioning. The ultrasonic device may be used to supplement other determinations of relative position and aspect derived from sensors or controls in each of the relevant devices. The ultrasonic device 12 may be used as a positioning input, or a safety stop, when the devices come closer than a pre-determined distance from each other.

The C-arm X-ray system 20 may be controlled by a control interface 2, which may also include the X-ray generator 3, or at least the high voltage power supply. Other devices that may be useful in diagnosis or treatment of a patient, such as a patient monitor 8 for monitoring vital signs, and a ventilator 32, may also be included. One or more display units 6 may be provided for visualizing the CT images and other data obtained from the various components of the equipment suite, including the status of devices.

The robots 27 and 60 may be controlled by a controller or processor 11, which receives data from a user interface 15, and which also may communicate with a DICOM system and with external devices over a network interface 80. The patient terminal 14 may be a notebook computer, or other processing device with which the demographic, history, diagnosis and/or therapy data of the patient can be recorded, recalled, and sent to and from the medical information management system of the hospital. This device may communicate with other devices by wireless techniques.

The sensor and robot portions of the treatment suite may be located in a therapy room, and some or all of the signal and data processing and data display may also be located in the treatment room; however, some or all of the equipment and functionality not directly related to the sensing or manipulating of the patient, may be remotely located. Such remote location is facilitated by high speed data communications on local area networks, wide area networks, and the Internet. The signals representing the data and images may be transmitted by modulation of representations of the data on electromagnetic signals such as light waves, radio waves, or signals propagating on wired connections.

The treatment suite may thus be located remotely from the specialists making the diagnosis and for determining or administering the appropriate course of treatment. Of course, the specialists may be present with the patient at times as well.

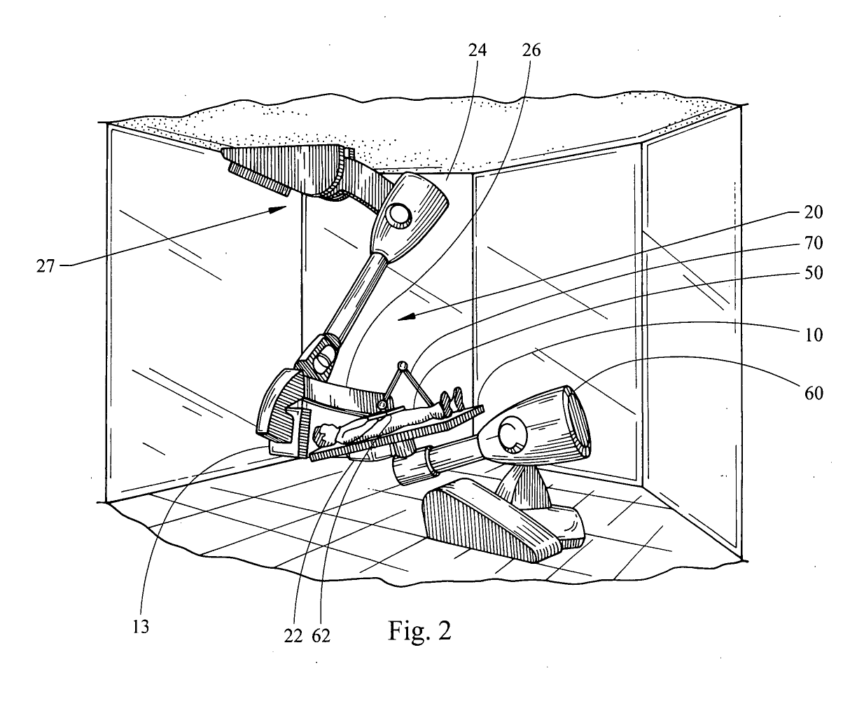

FIG. 2 is a perspective view of some of the equipment which may be in a treatment room. The robot 27 may be mounted to a ceiling and supports and positions the C-arm X-ray device 20 by an articulated arm 24, so that the C-arm 26 and the associated X-ray source 22 and detector 13 may be suitably positioned with respect to a patient 50 on a patient support apparatus 10. The patient support apparatus 10 may also be mounted to a robotic device 60, which is shown mounted to the floor of the room, so that, cooperating with the robotic device 27, the C-arm X-ray device may be located such that the projection X-ray image data to be used in synthesizing (computing) the computed tomography (CT) image may be obtained.

A third robotic arm 70 may be mounted to any one of the robots 26 or 60, or to the patient support apparatus 10. The arm is positionable so that a force applying surface 62 may be brought in contact with the patient 50. The robotic arm 70 may be controlled either automatically, semi-automatically or manually so as to bring the force applying surface 62 into contact with the patient body at a position suitable for the diagnostic protocol being performed. The amount of force, the sequence of operations, and the coordination with obtaining digital images of the patient are dependent on the specific diagnostic protocol being performed.

The third robotic arm 70 may be provided with a stowed position when not in use where the arm is folded against the associated robot or structure so that other equipment may be used. A force sensor (not shown), which may be a strain gauge or the like, may be provided to limit the amount of force that may be applied to the patient body.

Some or all of the data collected or processed by the treatment suite may be forwarded to another entity for use in diagnosis, billing and administrative purposes, or further image processing and storage using known interfaces such as DICOM (Digital Communications in Medicine) and SOARIAN, or special purpose or later developed data formatting and processing techniques. SOARIAN is a web-browser-based information management system for medical use, integrating clinical, financial, image, and patient management functions and facilitating retrieval and storage of patient information and the performance of analytic tasks (available from Siemens Medical Solutions Health Service Corporation, Malvern, Pa).

A method of diagnosing or treating a patient is disclosed, including: providing a projection X-ray radiographic apparatus, or other imaging apparatus, the apparatus being mounted to one or more robots mounted on surfaces of a room; providing a patient support apparatus, mounted to a robot, the robot being mounted on a surface of the room; orienting the radiographic apparatus with respect to a patient positioned on the patient support apparatus so as to obtain a sequences of radiographic images, suitable for synthesis of a computed tomography (CT) image of the abdominal area.

The robot manipulating the X-ray apparatus and the robot manipulating the patient support apparatus cooperatively position or reposition the patient and the X-ray apparatus so that image data consistent with a selected diagnosis or treatment protocol is obtained and processed. The positioning actions may be in accordance with a pre-determined examination or treatment protocol or controlled manually by an operator.

In another aspect, the method includes applying a controlled pressure to the patient abdomen using a robotic arm, the arm being controlled either in accordance with a pre-determined examination or treatment protocol or manually by an operator. The robotic arm may contact the patient either directly or indirectly through an attached pressure pad, and the robotic device is adapted to sense the amount of force being applied and to maintain the force below a pre-determined user or system determined threshold. The motions of the robotic arm may be coordinated with the operation of the X-ray apparatus so as to obtain images of the abdomen in accordance with the diagnostic protocol.

A method of work flow for diagnosis or treatment of abdominal illnesses is disclosed, including bringing the patient to a treatment room, providing a treatment suite suitable for diagnosing or treating abdominal illnesses; positioning the patient on a robotically controlled patient support apparatus; operating a robotically positioned imaging modality cooperating with the patient support apparatus such that the location and orientation of the one or more imaging modalities with respect to the patient is determined to obtain tomographic images of some of all of the abdomen in accordance with a diagnostic or treatment protocol.

In another aspect, various contrast agents may be administered to the patient during the examination or treatment, either as individually, simultaneously, or contemporaneously administered substances.

In yet another aspect, imaging data obtained by the imaging modality may be processed to produce any computed tomographic image, such as sections, 3-D and 4-D visualizations, and the like for viewing by medical personnel, and used to perform a diagnosis or a treatment without moving the patient to another examination or treatment room. Where a treatment is performed, the images may be used to visualize the position of treatment devices disposed internally to the patient, and used to automatically position the devices, or to provide information to be used by the medical personnel to position the devices manually or semi-automatically.

In a further aspect, the method includes performing a sequence of diagnostic procedures, or obtaining multiple diagnostic results from the same procedure without moving the patient to another room or patient support apparatus. The imaging modality data may be used for, for example, angiographic studies, soft tissue imaging, and hard tissue imaging. Alternatively, different imaging modalities, manipulated by one or more robots may be positioned with respect to the patient so as to obtain different types (e.g. acoustic and X-ray) of data (e.g. acoustic and X-ray). During the course of obtaining the data, the patient may remain on the patient support apparatus and the various ancillary equipment, such as EKG, bodily function monitors, and life support equipment, may remain connected to the patient.

In yet another aspect, the method includes providing a patient support apparatus that is removably and manipulably attached to a robot, and manipulating the patient support apparatus so as to position the patient appropriately for a selected diagnostic procedure or treatment. The patient support apparatus may be detached from the robot and attached to a post or holding apparatus so as to maintain the position of the patient. An imaging modality is provided that capable of being removably and manipulably attached to a robot. When not attached to the robot, the imaging modality may be attached to a post or a holding apparatus. The robot and the imaging modality are attached, and the imaging modality is positioned with respect to the patient by the robot. The imaging modality may be operated, and cooperate with, the robot so as to obtain one or more images of the patient which may be suitable for diagnosis or treatment of an illness or syndrome. The method may be performed by a single robot or by multiple robots, may include applying a controlled pressure to the patient abdomen using a robotic device, the device being controlled either in accordance with a pre-determined examination or treatment protocol, or manually by an operator.

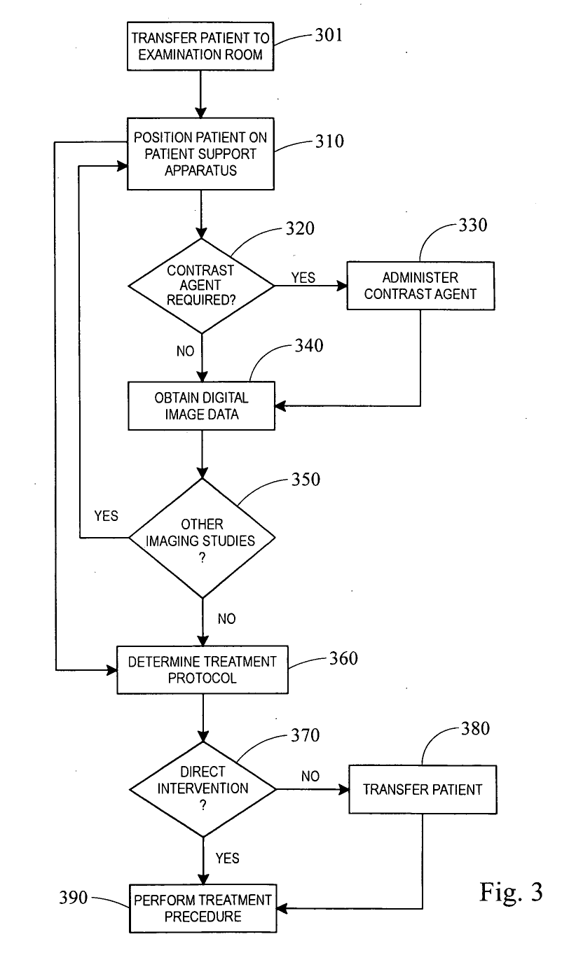

In an example, as shown in FIG. 3 the work flow of the method may include: bringing the patient to the examination room (301). The patient may then be positioned on the patient support apparatus, and intubated or attached to such other apparatus as may be necessary, such as an EKG, bodily function monitor, or the like, if such actions have not already been taken (step 310). A determination is made as to whether a contrast agent is to be administered prior to or during the collection of digital imaging data (step 320) and the contrast agent administered or scheduled for administration (step 330). Contemporaneously with the administration of the contrast agent, if a contrast agent is needed, the robots may be controlled so as to position the patient in accordance with the diagnostic or treatment protocol and digital imaging data is obtained (step 340). During the image data obtaining step 340, a robot having a force applying device may be used to apply pressure to the abdomen. Further, the patient may be repositioned prior to, or during the course of, gathering digital imaging data. After the collection of the digital imaging data, a determination is made as to whether additional imaging studies are needed (step 350). Should additional studies be needed, the work flow returns to step 320. After obtaining the digital imaging data, a diagnosis may be made to determine the appropriate treatment protocol (step 360). It will be understood by persons of skill in the art that step 360 may require interpretation of the digital imaging data obtained when the data has been processed into CT-like formats, consultation with colleagues, use of other diagnostic equipment and data bases and the like. Once the treatment protocol has been determined (step 360), a decision as to whether to perform a direct intervention while the patient remains in the treatment room or to transfer the patient to another room is made (step 370). If direct intervention is the selected method of treatment, the appropriate medications and treatment devices are used (step 390). The treatment may also use the imaging modalities used in step 340, the administration of contrast agents (step 330) and the positioning capabilities of the robots associated with steps 310 and 340 to position the patient, and to obtain imaging data as needed to assist in administration of the appropriate treatments. At any time during the work flow, a health care professional may choose to modify the sequence of steps, or omit certain steps as the medical circumstances may indicate.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

Although only a few examples of this invention have been described in detail above, those skilled in the art will readily appreciate that many modifications are possible without materially departing from the novel teachings and advantages of the invention. Accordingly, all such modifications are intended to be included within the scope of this invention as defined in the following claims.

What is claimed is:

1. A treatment unit, comprising:
a first robot to which an imaging modality configured to obtain projection digital imaging data is manipulably mounted;
a second robot to which a patient support apparatus is manipulably mounted;
a controller configured to control the first robot and the second robot to orient a patient captivated to the patient support apparatus so that digital images of the patient may be obtained by the imaging modality; and
a processor configured to synthesize tomographic images from the digital image data.

2. The unit of claim 1, wherein the imaging modality is a C-arm X-ray apparatus.

3. The unit of claim 1, wherein the imaging modality is removably attached to the first robot, and patient support apparatus is removably attached to the second robot.

4. The unit of claim 1, wherein the first robot and the second robot are the same robot.

5. The unit of claim 1, wherein at least one of the first or the second robots are mounted to one of a ceiling, a wall, or a floor.

6. The unit of claim 1, wherein the patient support apparatus is a gurney or a patient examination table.

7. The unit of claim 1, wherein the patient support apparatus is adapted to support the patient and positionable such that the patient may be placed in one of at least two positions selected from a supine, prone, upright or seated.

8. The unit of claim 1, wherein at least one of the imaging modality, the robot or the patient support apparatus has a collision avoidance sensor.

9. The unit of claim 1, further comprising a processor configured to synthesize tomographic soft-tissue images.

10. The unit of claim 1, further comprising a third robot having a manipulable arm and a forcer surface, wherein the controller cooperatively positions the forcer surface with respect to a patient to apply a force to a body part.

11. The unit of claim 10, wherein the third robot has a force sensor configured to limit a maximum force which may be applied to the body part.

12. The unit of claim 10, wherein the third robot is attached to one of the first robot, the second robot, or the patient support apparatus.

13. The unit of claim 1, wherein the robots are controlled by computer readable instructions stored on a removable or a remotely located machine readable media.

14. The unit of claim 1, wherein the imaging modality has a data interface in communication with a network.

15. The unit of 14, wherein the data interface is configured to transmit data by modulating information on a carrier wave.

16. The unit of claim 1, wherein the imaging modality is comprised of an ionizing radiation generator and a ionizing radiation detector, and the generator and the detector are mounted to the first robot.

17. A method of diagnosing or treating a patient, the method comprising:
providing a digital imaging modality;
mounting the digital imaging modality to a first robot, the first robot being mounted to a surface of a room;
providing a patient support apparatus;
mounting the patient support apparatus to a second robot; and
cooperatively orienting the digital imaging modality and a patient secured to the patient support apparatus so as to obtain a radiographic image of a patient abdomen, suitable for synthesis of a computed tomography (CT) image.

18. The method of claim 17, wherein the first and the second robots are the same robot.

19. A method of diagnosing or treating a patient, the method comprising:
bringing a patient to a treatment room;
placing the patient on a patient support apparatus manipulable by a first robot;
providing an imaging modality manipulable by a second robot;
cooperatively manipulating the patient support apparatus and the imaging modality to obtain digital image data of a patient abdominal region; and
preparing a computed tomographic image from the digital image data.

20. The method of claim 19, wherein two or more distinct digital imaging modalities are used to obtain digital images.

21. The method of claim 19, wherein a treatment is administered to the patient without moving the patient from the patient support apparatus.

22. The method of claim 20, further comprising:
determining an appropriate treatment based on the digital imaging data and administering the treatment while the patient remains on the patient support apparatus.

23. The method of claim 22, wherein the patient support apparatus remains attached to the first robot.

24. The method of claim 20, wherein at least two different contrast enhancement materials are administered to the patient while the patient remains on the patient support apparatus.

25. The method of claim 20, further comprising:
providing a third robot having a forcer configured to apply a force to a patient body part.

26. The method of claim 25, wherein the forcer cooperates with the imaging modality to apply the force during a portion of a digital data acquisition process.

27. The method of claim 25, wherein any of the first, second or third robots are the same robot.

28. A method of diagnosing an illness or treating a patient, the method comprising:
providing a robot;
providing a patient support apparatus removably and manipulably mountable to the robot;
providing an imaging modality removably and manipulably mountable to the robot;
attaching the patient support apparatus to the robot and manipulating the patient support apparatus so as to position a patient;
detaching the patient support apparatus from the robot;
attaching the imaging modality to the robot;
manipulating and operating the imaging modality to obtain digital image data of a patient; and
preparing a computed tomographic image from the digital image data.

29. The method of claim 28, wherein the illness or treatment relates to a medical condition of gastro-intestinal origin.
